(19) United States
(12) Patent Application Publication
(10) Pub. No.: US 2016/0374603 A1
(43) Pub. Date: Dec. 29, 2016

(54) ANALYTE TESTING DEVICES WITH LANCET ADVANCEMENT TRACKING AND COLOR TOUCH SCREEN USER INTERFACE

(71) Applicant: YOFIMETER, LLC, La Jolla, CA (US)

(72) Inventors: Gad Shaanan, La Jolla, CA (US); Marc Goldman, San Diego, CA (US); Jessica Shaanan, San Diego, CA (US)

(21) Appl. No.: 15/125,118

(22) PCT Filed: Mar. 12, 2015
(86) PCT No.: PCT/US2015/020283

§ 371 Date: Sep. 9, 2016

Related U.S. Application Data
(60) Provisional application No. 61/952,070, filed on Mar. 12, 2014.

(51) Int. Cl. A61B 5/157; A61B 5/00; A61B 5/151; A61B 5/145; A61B 5/15

(52) U.S. Cl. CPC A61B 5/157; A61B 5/14546; A61B 5/150412; A61B 5/15153; A61B 5/150961; A61B 5/7435; A61B 5/15087; A61B 5/15182; A61B 5/150801; A61B 5/14532; A61B 5/150175

(57) ABSTRACT

Analyte testing and monitoring methods, systems, and devices are disclosed. In one aspect, an analyte testing device includes an analyte sensor module configured to hold a sensor cassette structured to store analyte sensors, the analyte sensor module including an opening from which an analyte sensor advances to a testing position to expose at least a portion of the analyte sensor to outside of the analyte testing device; and a lancing module configured to hold a lancet cassette structured to store lancets and project the lancets to outside of the analyte testing device during a blood sample acquisition event to pierce a user's skin.

### CROSS REFERENCE TO RELATED APPLICATIONS

This patent document claims the benefit of priority of U.S. Provisional Patent Application No. 61/952,070, entitled “ANALYTE TESTING DEVICES WITH LANCET ADVANCEMENT TRACKING AND COLOR TOUCH SCREEN USER INTERFACE,” filed on Mar. 12, 2014. The entire disclosure of application 61/952,070 is incorporated herein by reference for all purposes.

### TECHNICAL FIELD

This patent document relates to analyte testing devices and related techniques.

### BACKGROUND

Analyte testing and monitoring devices play a critical role in modem diagnosis and management of health-related issues. An analyte, or component (in clinical chemistry), is a substance or chemical constituent that is of interest in an analytical procedure. For example, a sample of human blood, urine, and/or saliva can be tested for glucose, fructosamine, hematocrit, hemoglobin blood oxygen saturation, lactates, iron, pH, cholesterol, liver enzymes (e.g., aspartate aminotransferase (AST), alanine aminotransferase (ALT), alkaline phosphatase (ALP)/gamma glutamyl transferase (GOT), lactate dehydrogenase (LDH), bilirubin, etc.), hormones, and/or other compounds.

### SUMMARY

Techniques, systems, and devices are disclosed for analyte monitoring having a blood sample acquisition module and color touch screen interactive display unit to provide a patient interface for analyte testing.

In one aspect, an analyte testing device includes an analyte sensor module configured to hold a sensor cassette structured to store analytc sensors, the analyte sensor module including an opening from which an analyte sensor advances to a testing position to expose at least a portion of the analyte sensor to outside of the analyte testing device; a lancing module configured to hold a lancet cassette structured to store lancets and project the lancets to outside of the analyte testing device during a blood sample acquisition event to pierce a user's skin; a sensor module in communication with the lancing module, the sensor module including one or more sensors to detect one or more of a lancing projection operation of the device, presence or absence of a lancet in the lancet cassette, or a position or movement of the lancet cassette; a processing unit in communication with the sensor module and configured to determine information associated with the detected lancing projection operation, presence or absence of the lancet in the lancet cassette, or the position or movement of the lancet cassette; and an interactive display unit in communication with the processing unit and including a color touch screen display to receive user inputs from a user and to display the determined information to the user of the device.

This and other aspects and their implementations are described in detail in the drawings, the description and the claims,

### BRIEF DESCRIPTION OF THE DRAWINGS

FIG. 1 shows a diagram of an analyte testing device including a blood sample acquisition module of the disclosed technology and color touch screen to provide a patient interface for analyte testing.

FIG. 2 shows a schematic illustration of an exemplary lancet cassette of the blood sample acquisition module of the disclosed technology.

FIG. 3 shows a schematic illustration of the exemplary lancet cassette and sensors of the blood sample acquisition module.

FIG. 4 shows an illustrative schematic diagram depicting interaction of the exemplary sensors and lancet cassette in an exemplary lancing operation of the analyte testing device.

FIG. 5 shows a schematic illustration of the exemplary lancet cassette and sensors of the blood sample acquisition module in communication with a processing unit and an exemplary interactive color touch screen display of the device.

### DETAILED DESCRIPTION

Devices, systems and technologies described in this patent document can be implemented to measure properties of analytes such as glucose concentration in blood samples. Various glucose meters and lancing devices on the market today tend to involve multiple devices, components, and supplies, and often require numerous steps to monitor glucose levels. When such devices are designed for patient uses outside the clinical settings or hospitals, the complexity in operating the devices and performing the measurements may lead to patient operation errors and false data and may also cause patient frustration and reluctance in routine use of such devices. For example, some glucose monitoring systems may require numerous steps involving reading a test strip, readying a lancet, using the lancet, putting blood on the test strip and inserting the strip into the glucose meter, reading data from a meter, recording the data in a journal and remembering to bring the journal to the next doctor visit, and then putting away the strip and lancet packages, disposing of loose components, and storing the glucose meter. Thus, it would be beneficial to patients, caregivers, and payers to reduce steps, consolidate devices, and simplify user interfaces for monitoring analytes, e.g., such as glucose in the blood.

Devices, systems, and techniques are disclosed for analyte testing having a blood sample acquisition module to ensure a single sample-taking by a user and interactive display unit to provide a patient interface for analyte testing.

While the disclosed embodiments are described herein primarily based on glucose monitoring to facilitate understanding of the underlying concepts, it is understood that the disclosed embodiments can also include monitoring of other analytes that include, but are not limited to, fructosamine, hematocrit, hemoglobin blood oxygen saturation, lactates, iron, pH, cholesterol, liver enzymes (e.g., AST, ALT, ALP/GGT, LDH, bilirubin, etc.), hormones, and other compounds. For example, other biomolecular substances can also be monitored using analytical monitoring techniques of the disclosed embodiments, which include, but are not limited to, nucleic acids, lipids, carbohydrates, peptides, proteins, enzymes, hormones, antibodies, glycoproteins, glycolipids, organelles, endotoxins, and viruses, among other biological materials and biomarkers.

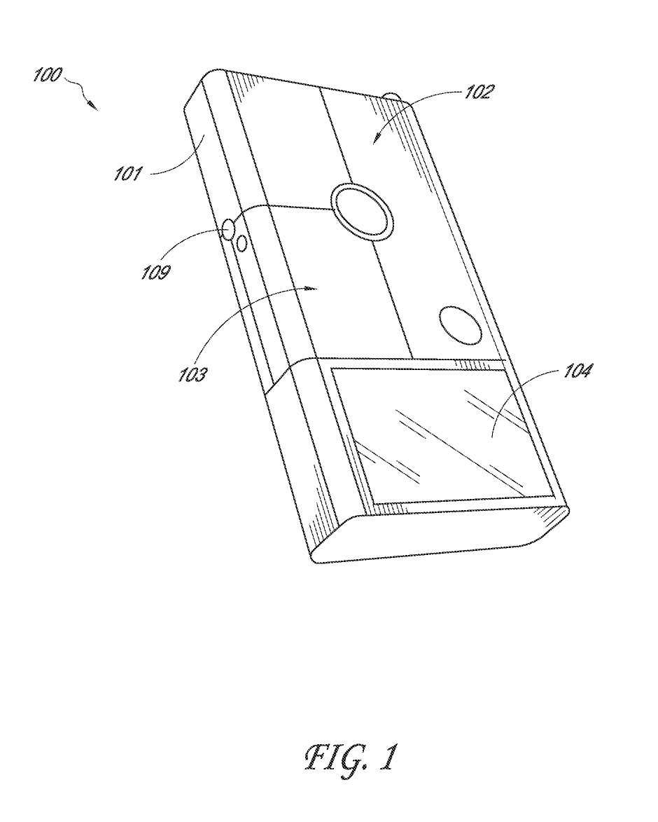

FIG. 1 shows a diagram of an exemplary embodiment of an analyte testing device 100 that includes one exemplary implementation of a blood sample acquisition module and interactive color touch screen display 104 to provide a patient interface for analyte testing. For example, the color touch screen display 104 can present colorful, visibly distinct icons allowing ease of direct touch by the user to receive user inputs and to enhance the user navigation of controls. The color touch screen display 104 also provides a viewing of data and information provided by the analyte testing device. The color touch screen display 104 acts as an interactive user interface for user interactions and viewing. For example, the colors of icons, graphs, and other displayed features presented by the interactive color touch screen display 104 can represent and distinguish analyte level data and other patient health information in corresponding colors for enabling the user to distinguish between information and data types and levels on the same display screen.

As shown in FIG. 1, the device 100 includes an external housing or device casing 101 having a lancet projection opening 109. The device 100 includes an analyte sensor module 102 and a lancing module 103 contained within separate cavities located within the device 100. The analyte sensor module 102 is configured to hold a sensor cassette structured to store analyte sensors (e.g., test strips), in which the analyte sensor module 102 includes an opening from which an analyte sensor advances to a testing position to expose at least a portion of the analyte sensor to outside of the analyte testing device 100 during a blood sample acquisition event. The lancing module 103 configured to hold a lancet cassette structured to store lancets and project (e.g., sometimes referred to as “fire”) the lancets to outside of the analyte testing device 100 during a blood sample acquisition event to pierce a user's/patient's skin and draw blood. In some embodiments, the blood sample acquisition module and interactive color touch screen display 104 of the analyte testing device 100 can be incorporated and implemented according to analyte testing device embodiments described in U.S. Patent Publication No. 2013/0245392, entitled “ANALYTE TESTING DEVICES,” and PCT Publication No. WO2014022711, entitled “USER INTERFACE FOR ANALYTE MONITORING SYSTEMS,” the entire content of which are incorporated by reference as part of the disclosure in this patent application.

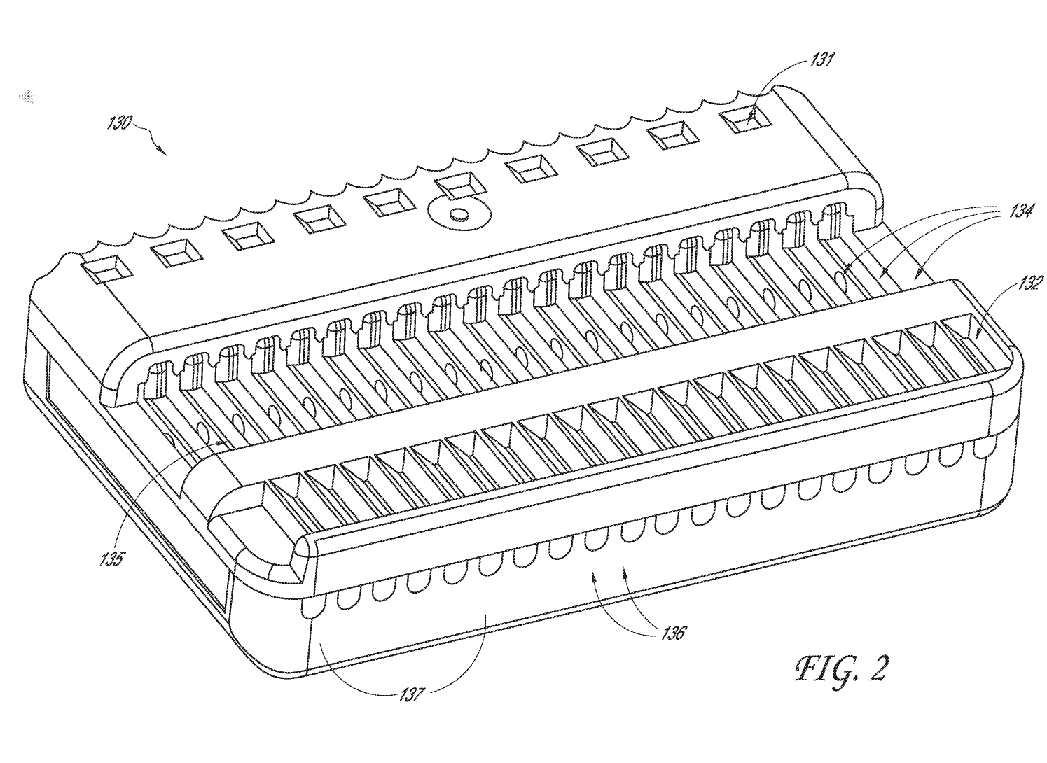

FIGS. 2-5 show schematic illustrations depicting the disclosed blood sample acquisition module and touch screen interactive display unit incorporated in the analyte testing device 100.

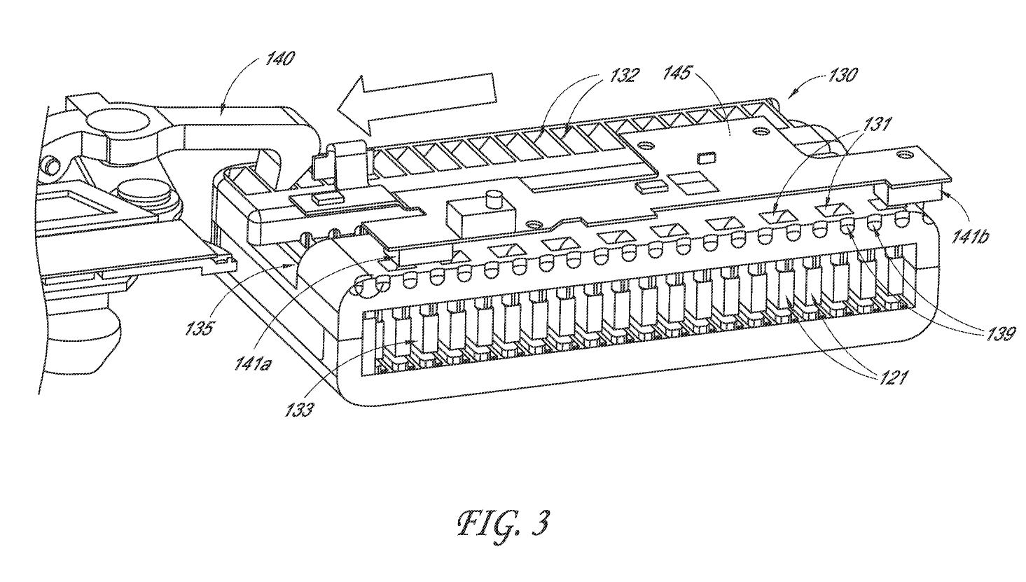

As shown in the schematic illustration of FIG. 2, the lancing module 103 of the analyte testing device 100 of FIG. 1 can be configured to include a lancet cassette 130. The lancet cassette 130 includes a channel region 135 along the exterior of the internal side of the cassette 130. The channel 135 provides a region in which a lancet depth adjustment component of the lancing module 103 can pass through such that a step of the series of steps can be aligned in the firing path of a lancet to be fired. The lancet cassette 130 includes an advancing rack 132 (e.g., configured as an array of toothed indentations) along the exterior of the internal side of the lancet cassette 130. The toothed advancing rack 132 provides indentations into the exterior that can engage a spring loaded pawl of the lancing module 103 for the advancement of the lancet cassette 130 in the linear direction during a lancing operation of the blood sample acquisition module. The lancet cassette 130 includes a covering layer 137 (e.g., such as a film) along the exterior of the bottom side of the lancet cassette 130 (e.g., which is shown to wrap around the side(s) of the cassette). The covering layer 137 provides a protective covering over openings 136 to the lancet chambers to protect the lancet from contaminations and maintain a sterile environment within the chambers, as well as bonds the top and bottom of the lancet cassette 130 together. The openings i36 provide the holes of the lancet cassette 130 from which the piercing portion of a lancet is fired from the lancing module 103 and out of the device 100 to implement a blood sample acquisition event. The lancet cassette 130 includes open troughs 134 within the channel 135 to allow a spring arm of the lancing module 103 to return to its initial position after a lancet has been fired. The lancet cassette 130 includes a switch-contact flag or well 131 configured on the exterior of the internal side of the cassette 130 and positioned in parallel with the open troughs 134 and/or the toothed advancing rack 132. The switch-contact flag 131 provides a structure that can trigger a detection event by one or more sensor switches (shown in FIG. 3 and FIG, 5) to detect an implementation of a lancing operation and/or the presence or absence of a lancet in the lancet cassette 130. In some implementations, for example, the switch-contact flag 131 can be structured to have a pitch (or width) double the pitch or width) of a tooth indention of the toothed advancing rack 132. In other implementations, for example, the switch-contact flag 131 can be structured to have a pitch (or width) equal to the pitch or width) of a tooth indention of the toothed advancing rack 132, or triple or greater the size of a tooth indention.

As shown in the schematic illustration of FIG. 3, the lancet cassette 130 is shown in an exemplary lancing operation of the lancing module 103. In this exemplary schematic illustration, the opposite side of the lancet cassette 130 is shown, displaying a portion of the lancets 121, in which a contact side of each lancet 121 is exposed out of an open region 133 of a top side lancet cassette 130 such that a firing hammer component of the lancing module 103 can contact and move the lancet 121, e.g., during a lancing operation of the blood sample acquisition module. The lancet cassette 130 includes detents 139 along an edge of the top and internal side of the cassette 130 to retain an indexed position of the lancet cassette after advancement. For example, the movement of a sliding component 140 of the lancing module 103 in the linear direction advances the lancet cassette 130 by one lancet position that is indexed by the detent 139 such that a lancet 121 that is to be fired is placed in a position aligned in the firing path of the firing hammer component for projection of that lancet. In the example shown in FIG. 3, the sliding component 140 of the lancing module 103 can be configured as a mechanical pawl component to provide an advancing pawl mechanism.

As shown in the schematic illustration of FIG. 3, the blood sample acquisition module includes two or more sensors to interact with the lancing module 103, including sensor switch 141a and sensor switch 141b, attached to a sensor positioning backbone 145, which can be attached to the internal housing structure of the device 100. The sensor positioning backbone 145 provides a securement to maintain the sensor switches 141a and 141b in a stationary position relative to the lancet cassette 130, which allows the sensor switches 141a and 141b to detect movement, position, and other information about the lancet cassette 130 and the lancets 121. For example, the sensors 141a and 141b can detect the presence of a cavity/flat surface (e.g., switch contact flag 131 and the exterior casing of the lancing cassette 130) on the lancet cassette 130 to confirm the movement (e.g., forward) of the lancet cassette 130. In sonic implementations, for example, the sensors 141a and 141b can be optical sensors to optically detect the presence of a cavity/flat surface. For example, the sensor switch 141a can be configured to validate an advancement event of the lancet cassette 130 in the cavity of the device 100 containing the lancing module 103, in which such advancement aligns a lancet to be fired in a readied position (e.g., firing path) to be fired by the firing hammer component for projection of that lancet from the lancet projection opening 109. The sensor switches 141a and 141b engages with the side of the lancet cassette 130 having the switch-contact flags 131. The sensor switch 141b can be configured to confirm the lancet cassette 130 is loaded at its initial position.

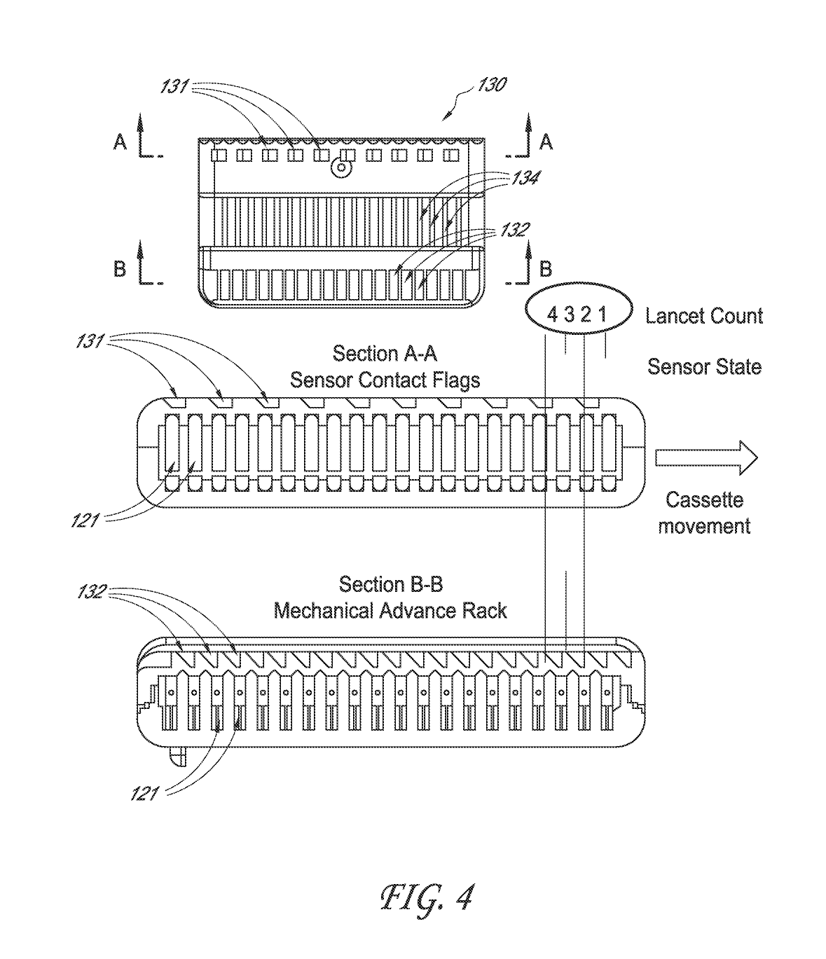

FIG. 4 shows an illustrative schematic diagram depicting interaction of the sensors 141 and the lancet cassette 130 in an exemplary lancing operation of the analyte testing device 100. For example, in one implementation of the device 100 to acquire a blood sample using the lancing module 103, a user can initiate a new analyte test by actuating the external slide button of the device 100 (e.g., such as the sliding button including the cocking button 1830 and eject button 1835 that move translationally along the sliding track 1839 of the exemplary embodiment of the analyte testing device depicted as the device 1800 in U.S. Patent Publication No. 2013/0245392). The external slide button is linked Co the internal mechanical pawl component 140. For example, the pawl 140 can be configured on a track that recirculates linear motion for every actuation. The pawl 140 engages onto a single tooth of the toothed advancing rack 132 to allow incremental indexing of the lancet cassette 130.

In some implementations, for example, the pawl 140 can be configured to have a spring to allow clutching to the lancet cassette 130 when it recirculates to the nominal start position. In the example shown in FIG. 4, the switch-contact flags 131 of the lancet cassette 130 that engage to the sensor switch 141 are distributed at two times the pitch of the toothed advancing rack 132 so that the sensor state (e.g., open/closed) of the sensor 141 changes for each new lancet advancement. For example, in an initial position with a fully-loaded lancet cassette 130 with a total number of lancets 121, the sensor switch 141b can provide a binary signal indicating the lancet cassette 130 is in its initial position, and the sensor switch 141a can provide a binary signal indicating a closed sensor state, e.g., corresponding to a lancet count “1”, which ultimately indicates that the lancet cassette has all of the lancets 121 in the cassette 130. Upon firing the first lancet from the device 100, the actuator mechanism can cause the lancet cassette 130 to move (e.g., via the mechanical pawl component 140), such that the sensor switch 141b is no longer aligned with the cassette 130 (thus indicating that the lancet cassette 130 is not in its initial position) and the sensor switch 141a is switched to another sensor state (e.g., open) due to its new alignment with a switch-contact flags 131, e.g., and thereby changing the lancet count to “2”.

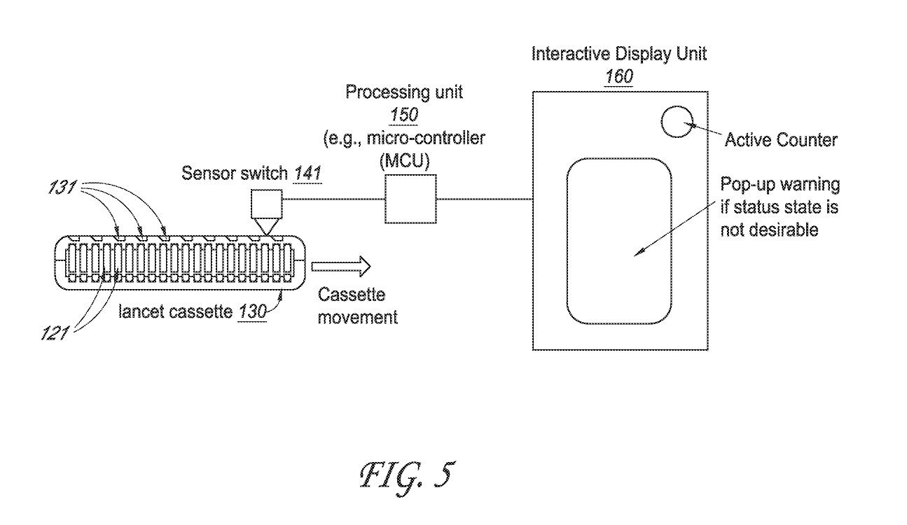

FIG. 5 shows a schematic illustration of the blood sample acquisition module in communication with a processing unit 150 and the interactive display unit 160 (in communication with the interactive color touch screen display 104) of the device 100. The processing unit 150 can include a processor to process data and a memory unit in communication with the processor to store data. In some implementations, for example, the processing unit 150 can include a microcontroller (MCU). The memory unit can, for example, include processor executable code, which when executed by the processor, configures the device to perform various operations, such as receiving information, commands, and/o data, processing information and data, and transmitting or providing information/data to another entity or to a user.

The interactive display unit 160 can be implemented to keep and/or display the count of unused lancets remaining in the lancet cassette 130. For example, in an exemplary ‘new test’ scenario, a user actuated button of the display unit 160 can contact a switch to confirm new test has initiated. The processing unit 150 (e.g., MCU) can look for a state change with the sensor switch 141 to confirm that a lancet 121 has advanced. If no change is detected, for example, the processing unit 150 can change the display image on the color touch screen display 104 to show a “pop-up” warning, which can require user validation. If change is confirmed, for example, the active counter can be modified.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter affecting a machine-readable propagated signal, or a combination of one or more of them. The term “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor ironware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination, Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. An analyte testing device, comprising:
analyte sensor module configured to hold a sensor cassette structured to store analyte sensors, the analyte sensor module including an opening from which an analyte sensor advances to a testing position to expose at least a portion of the analyte sensor to outside of the analyte testing device;
a lancing module configured to hold a lancet cassette structured to store lancets and project the lancets to outside of the analyte testing device during a blood sample acquisition event to pierce a user's skin;
a sensor module in communication with the lancing module, the sensor module including one or more sensors to detect one or more of a lancing projection operation of the device, presence or absence of a lancet in the lancet cassette, or a position or movement of the lancet cassette;
a processing unit in communication with the sensor module and configured to determine information associated with the detected lancing projection operation, presence or absence of the lancet in the lancet cassette, or the position or movement of the lancet cassette; and
an interactive display unit in communication with the processing unit and including a color touch screen display to receive user inputs from a user and to display the determined information to the user.

2. The device as in claim 1, wherein the display unit includes an touch screen display.

3. The device as in claim 1, wherein the processing unit is in communication with one or both of the lancing module and the analyte sensor module.

4. The device as in claim 3, wherein the processing unit is configured to perform one or more of the following:
correlate individual instances of data obtained from the analyte sensor or information derived from data out of the analyte testing device with (a) time stamps or (b) user entered information including speech or text,
evaluate data obtained from the analyte sensor or information derived from data out of the analyte testing device, and send a notification to a recipient,
keep track of inventory of lancets and analyte sensors, both within and outside the device, wherein the processing unit is configured to reorder inventory of lancets and analyte sensors, or
produce a prompt to direct a user to use the device according to at least one of a selected time or a selected time interval.

5. The device as in claim l, wherein the lancet cassette is structured to include:
an advancing rack comprising an array of toothed indentations formed on a first side of the exterior of the lancet cassette, the advancing rack structured to engage a pawl mechanism of the device for the advancement of the lancet cassette in a linear direction during a lancing operation, and
an array of sensing wells on the first side to provide a structure to cause a detection event by the sensor unit, the array of sensing wells positioned in parallel with the toothed indentations of the advancing rack,
wherein the array of sensing wells are structured to have a pitch sized at least double of a pitch size of the tooth indentions of the advancing rack.

6. The device as in claim l, wherein the lancet cassette is structured to include:
lancet chambers to store the lancets, the lancet chambers each having a projection opening to allow a piercing portion of the lancet to project through and outside of the device, and
a covering layer formed over a portion of the lancet cassette having the projection openings to protect the lancet chambers from contaminations and maintain a sterile environment within the chambers.

7. A method for testing an analyte using an analyte testing device,
advancing an analyte sensor from an opening in an analyte sensing module to a testing position, wherein at least a portion of the analyte sensor is outside of the analyte testing device when at the testing position, wherein the analyte sensing module is configured to hold a sensor cassette structured to store analyte sensors;
projecting a lancet from a lancing module to outside of the analyte testing device, wherein the lancing module is configured to hold a lancet cassette structured to store lancets;
performing a blood sample acquisition event using the lancing module;
detecting one or more of a lancing projection operation of the device, presence or absence of a lancet in the lancet cassette, or a position or movement of the lancet cassette, wherein the detecting is performed by one or more sensors of a sensing module in communication with the lancing module;
determining information associated with the detected lancing projection operation, presence or absence of the lancet in the lancet cassette, or the position or movement of the lancet cassette, wherein the determining is performed by a processing unit in communication with the sensor module; and
displaying the determined information on an interactive display unit, wherein the interactive display unit is in communication with the processing unit.

8. The method of claim 7, further comprising receiving user inputs from a user, wherein the receiving is performed by the interactive display unit, wherein the interactive display unit includes a color touch screen display;

9. The method of claim 7, wherein the display unit includes an LCD touch screen display.

10. The method of claim 7, wherein the processing unit is in communication with one or more or both of the lancing module and the analyte sensor module.

11. The method of claim 10, further comprising performing one or more of the following:
correlating individual instances of data obtained from the analyte sensor or information derived from data out of the analyte testing device with (a) time stamps or (b) user entered information including speech or text,
evaluating data obtained from the analyte sensor or information derived from data out of the analyte testing device, and sending a notification to a recipient,
keeping track of inventory of lancets and analyte sensors, both within and outside the device, wherein the processing unit is configured to reorder inventory of lancets and analyte sensors, or
producing a prompt to direct a user to use the device according to at least one of a selected time or a selected time interval.

12. The method of claim 7, further comprising engaging an advancing rack to a pawl mechanism of the device for the advancement of the lancet cassette in a linear direction during a lancing operation, wherein the advancing rack comprises an array of toothed indentations formed on a first side of the exterior of the lancet cassette.

13. The method of claim 12, wherein the analyte testing device includes an array of sensing wells on the first side to provide a structure to cause a detection event by the sensor unit, the array of sensing wells positioned in parallel with the toothed indentations of the advancing rack,
wherein the array of sensing wells are structured to have a pitch sized at least double of a pitch size of the tooth indentions of the advancing rack.
